(19) United States
(12) Patent Application Publication
(10) Pub. No.: US 2022/0226203 A1
(43) Pub. Date: Jul. 21, 2022

(54) SKIN-ATTACHABLE THIN FILM PATCH BASED ON A WATER-SOLUBLE NON-WOVEN FABRIC

(71) Applicant: WOOREEMNS CO.,LTD., Ansan-Si Gyeonggi-do (KR)

(72) Inventors: Young Bin SUNG, Hwaseong-Si Gyeonggi-do (KR); Won Youl SEO, Siheung-Si Gyeonggi-do (KR)

(21) Appl. No.: 17/612,937

(22) PCT Filed: May 21, 2020
(86) PCT No.: PCT/KR2020/006621

§ 371 Date: Nov. 19, 2021

(30) Foreign Application Priority Data
May 21, 2019 (KR) 10-2019-0059240

(51) Int. Cl. A61K 8/02; A45D 44/00; A61K 9/70; A61Q 19/00

(52) U.S. Cl. CPC A61K 8/0208; A45D 44/002; A61K 8/0212; A61Q 19/00; A61K 9/703

(57) ABSTRACT

The present invention is a thin film patch to be attached to the skin and used for skin treatment or skin care. In particular, a water-soluble polymer compound is formed by means of melt spinning, melt blowing or electrospinning, into a water-soluble non-woven fabric of which the average diameter of fiber is 5 μm or less. The water-soluble non-woven fabric is coated with a coating agent comprising an active ingredient with a skin treatment or skin care effect; therefore, a thin film having the total thickness of 50 μm and the basis weight of 20 gsm or less is formed, wherein the water-soluble non-woven fabric has an electrically conductive material deposited thereon to have electrical conductivity and is connected to an electrode so that a current can be applied thereto.

### TECHNICAL FIELD

The present invention relates a skin-attachable patch attached to skin to deliver an active ingredient for treatment or a skin care effect, and, more particularly, to a thin film patch made of a water-soluble non-woven fabric having a conductive material deposited thereon, as a base material.

### BACKGROUND ART

In general, a patch is configured to form an adhesive film layer using a hot-melt adhesive or a solvent-type adhesive on a transparent urethane film, or have skin adhesion by forming an adhesive layer on a non-woven fabric, and by mixing various therapeutic ingredients or an active ingredient, or adding an additional layer thereto, the patch is covered with released paper (see Patent Document 1).

However, since all these patches are thick, most of the patches, when they are used for a long time on the skin, involve skin side effects.

Hydrogels have been developed and known to improve the above-described disadvantage (see Patent Document 2). Such hydrogels are configured such that hydrophilic polymers have a network structure through hydrogen bonding or van der Waals bonding, thereby having swelling properties by water. Accordingly, the hydrogels become soft and stretchable and have adhesion, and are expanding their application to the medical and cosmetics fields due to their excellent biocompatibility.

Various types of hydrogels made by mixing a natural polymer such as chitosan and polyvinyl alcohol (PVA) with excellent adhesion and mechanical properties have been proposed.

Since the hydrogels were difficult to implement in the form of a thin film due to swelling, when the hydrogels were attached to the skin, they gave the user a feeling of weight, and the user can also feel stuffy due to their lack of air permeability.

### PRIOR ART DOCUMENT

[Patent Document]
(Patent Document 1) KR10-1154327B1
(Patent Document 2) KR10-1004329B1

### DISCLOSURE

### Technical Problem

The present invention is configured to solve the disadvantages of the conventional patch. The object of the present invention is to provide a skin-attachable thin film patch with an enhanced adhesive force, which, when attached to the skin, has a thin film structure with no foreign substances and which reduces stuffiness caused by blocking skin.

Another objective of the present invention is to provide a skin-attachable thin film patch which has increased skin permeability of the active ingredient of cosmetic liquid impregnated in the patch.

### Technical Solution

To this end, a thin film patch of the present invention includes: a base material; a water-soluble non-woven fabric formed over the base material; a coating liquid applied to the water-soluble non-woven fabric; and released paper which protects the coating liquid.

The water-soluble non-woven fabric may be made of water-soluble fiber by means of any one of melt spinning, melt blowing and electrospinning.

If a water-soluble non-woven fabric material of the present invention is suitable for medical or cosmetic use, it does not have specific limitation; however, a synthetic polymer such as polyvinyl alcohol, polyethylene oxide and polyvinylpyrrolidone, or modified sugar ingredients, such as pullulan and levan, may be preferably used.

If a thickness of the thin film is below 50 μm after the coating liquid is applied to the non-woven fabric, the non-woven fabric does not give the feeling of something foreign when attached to the skin and has sufficient transparency to make it difficult to visually identify the skin beneath it.

Therefore, in the present invention, in order to ensure that the thickness of the thin film has the aforementioned characteristics, the non-woven fabric used in the thin film patch of the present invention should have the basic weight of 20 gsm or less and an average diameter of 5 micrometers or less of fiber constituting the non-woven fabric. Preferably, the non-woven fabric should have the basic weight of 10 to 20 gsm and an average diameter of fiber of 1 to 5 μm.

Herein, when the basis weight is 10 gsm or less, the amount of the coating liquid impregnated into the thin film is not sufficient, and when the fiber diameter is too small, the penetration time of the coating liquid is too long to provide a slow production rate.

The coating liquid includes 20 to 50% by weight of a moisturizer, 10 to 40% by weight of purified water, 10 to 30% by weight of a thickener, 5 to 10% by weight of oil, and 2% by weight or less of additives.

The moisturizer imparts a moisturizing effect to the skin as a main ingredient of the coating liquid. Only the moisturizer which has no skin irritation, like glycerin and diglycerin, and that has been used before, can be used. The moisturizer wraps around the water-soluble non-woven fabric to prevent dissolution by moisture contained in the coating liquid and provides adhesion to the skin.

Since the thickener is used to control the viscosity of the coating liquid, and the thickener is used to impart a gel shape, only those thickeners that are not harmful to skin use, such as starch or cellulose gum, should be selected and used.

Purified water is used to provide moisture as one of the most important ingredients of the patch, and is mixed into the coating liquid by including numerous water-soluble active ingredients such as beta-glucan derived from mushrooms.

The oil serves to protect the skin along with a fat-soluble active ingredient, and as the oil is mixed with coating liquid, it delivers active ingredients to the skin.

Other additives produce the coating liquid by adding an antibacterial agent such as hexanediol, an odor such as lavender oil or propofol, and other necessary additives.

When producing the coating liquid, if the purified water containing the water-soluble active ingredient exceeds 40% by weight, this leads to damaging the water-soluble nonwoven fabric, and the thin film is difficult to form; therefore, the coating liquid should be carefully produced.

The amount of the coating liquid applied to the water-soluble non-woven fabric may be increased up to 10 times the basic weight of the non-woven fabric; however, the non-woven fabric may be preferably coated so that the amount of the coating liquid has 5 to 8 times the base weight. If the amount of the coating liquid is less than 4 times, the amount of coating liquid is too small to work on the skin, and if the amount of the coating liquid is more than 8 times, the completion of the product will be reduced due to the excessive amount of the coating liquid.

Another feature of the present invention is to provide a skin-attachable thin film patch that includes: a water-soluble non-woven fabric; and a coating liquid which has at least one functional substance and is applied to the water-soluble non-woven fabric, in which the water-soluble non-woven fabric has an electrically conductive material deposited thereon to have electrical conductivity, and an electrode is included in the water-soluble non-woven fabric to which electrical conductivity is applied.

When a current is applied to the thin film patch through an electrode in a state when the thin film patch is attached to the skin, the activity of the active ingredient applied to the water-soluble non-woven fabric increases to enhance skin permeability.

### Advantageous Effects

In the present invention, a patch of a thin film may be provided using a water-soluble nonwoven fabric, and, since an essence produced by dissolving a water-soluble active ingredient in water and dissolving a fat-soluble active ingredient in glycerin or oil may be coated and applied to a product, the product can be easily produced.

In the present invention, the current applied to the thin film patch electrically and/or thermally stimulates the skin and the active ingredient, thereby increasing the skin penetration of the active ingredient applied to the patch.

### MODE FOR THE INVENTION

In what follows preferable embodiments of the present invention are described.

A thin film patch of the present invention includes: a base material; a non-woven fabric formed over the base material and made of water-soluble fiber; a coating liquid applied to the water-soluble non-woven fabric; and released paper that protects the coating liquid.

The water-soluble fiber is formed by means of melt spinning, melt blowing, or electrospinning, into the water-soluble non-woven fabric.

If a water-soluble non-woven fabric material of the present invention is suitable for medical or cosmetic use, it does not have specific limitations; however, synthetic polymers such as polyvinyl alcohol, polyethylene oxide and polyvinylpyrrolidone, or modified sugar ingredients, such as pullulan and levan, may be preferably used.

If a thickness of the thin film is less than 50 μm after the coating liquid is applied to the non-woven fabric, the non-woven fabric does not give the feeling of something foreign attached to the skin and has sufficient transparency to make it difficult to visually identify the skin beneath it.

Therefore, in the present invention, in order to ensure that the thickness of the thin film has the above characteristics, the non-woven fabric used in the thin film patch of the present invention should have the basic weight of 20 gsm or less and an average diameter of 5 micrometers or less of fiber constituting the non-woven fabric. Preferably, the non-woven fabric should have the basic weight of 10 to 20 gsm and an average diameter of fiber of 1 to 5 μm.

Herein, when the basis weight is 10 gsm or less, the amount of the coating liquid impregnated into the thin film is not sufficient, and when the fiber diameter is too small, the penetration time of the coating liquid is too long to provide a slow production rate.

The coating liquid includes 20 to 50% by weight of a moisturizer, 10 to 40% by weight of purified water, 10 to 30% by weight of a thickener, 5 to 10% by weight of oil, and 2% by weight or less of additives.

The moisturizer imparts a moisturizing effect to the skin as a main ingredient of the coating liquid. Only the moisturizer which has no skin irritation, like glycerin and diglycerin, and that has been used before, can be used. The moisturizer wraps around the water-soluble non-woven fabric to prevent dissolution by moisture contained in the coating liquid and provides adhesion to the skin.

Since the thickener is used to control the viscosity of the coating liquid, and the thickener is used to impart a gel shape, only the thickeners that are not harmful to skin use, like starch or cellulose gum, should be selected and used.

Purified water is used to provide moisture as one of the most important ingredients of the patch, and is mixed into the coating liquid by including numerous water-soluble active ingredients such as beta-glucan derived from mushrooms.

The oil serves to protect the skin along with a fat-soluble active ingredient, and as the oil is mixed with coating liquid, it delivers active ingredients to the skin.

Other additives produce the coating liquid by adding an antibacterial agent such as hexanediol, an odor such as lavender oil or propofol, and other necessary additives.

When producing the coating liquid, if the purified water containing the water-soluble active ingredient exceeds 40% by weight, this leads to damaging the water-soluble nonwoven fabric and makes it difficult to form the thin film; therefore, the coating liquid should be carefully produced.

The amount of the coating liquid applied to the water-soluble non-woven fabric can be increased up to 10 times the basic weight of the non-woven fabric; however, the non-woven fabric may be preferably coated so that the amount of the coating liquid has 5 to 8 times the base weight. If the amount of the coating liquid is less than 4 times, the coating liquid is too small to work on the skin, and if the amount of the coating liquid is more than 8 times, the completion of the product can be reduced due to the excessive amount of the coating liquid.

Another embodiment of the present invention includes: a water-soluble non-woven fabric; a coating liquid which has at least one functional substance and is applied to the water-soluble non-woven fabric, the water-soluble non-woven fabric has electrical conductivity by depositing an electrically conductive material containing copper, silver or gold, and an electrode is included in the water-soluble nonwoven fabric to which the electrical conductivity is applied.

The water-soluble non-woven fabric is preferably produced by means of electrospinning of polyvinyl alcohol. However, the water-soluble non-woven fabric of the present invention is not limited thereto and is applied to a water-soluble non-woven fabric.

### Embodiment 11

A water-soluble polyvinyl alcohol (PVA) non-woven fabric having a basic weight of 20 gsm is prepared by means of electrospinning of polyvinyl alcohol (PVA) on the base material.

Next, the coating liquid is produced by mixing 60% of glycerin, 20% of cellulose gum, 10% of coconut oil, 8.5% of purified water and 1.5% of flavoring and other additives.

The coating liquid produced as described above is applied over the water-soluble polyvinyl alcohol non-woven fabric to prepare a thin film with the thickness of 50 μm and the basic weight of 120 gsm.

Then, the release paper is covered on an upper surface of the polyvinyl alcohol non-woven fabric.

The process of attaching the thin film patch produced according to Embodiment 1 to the skin is as follows.

A side of the thin film patch from which the released paper was removed is attached and evenly rubbed to the skin, and the base material then is removed from the side. Since the attached thin film patch is transparent, it is not easily identified whether or not it is attached to the skin, and since the thin film patch is light, there is no feeling of something foreign attached to the skin. Moreover, since the patch of the present invention is based on a water-soluble non-woven fabric, the patch is removable from the skin when washed with water after use.

### Embodiment 2

In Embodiment 1 above, the product was produced in the same process except that the basic weight of the PVA non-woven fabric was 10 gsm, while the essence coating amount was 80 gsm.

Since the thin film patch produced through this embodiment is thinner than the patch of Embodiment 1, the thin film patch gives a light sensation.

What is claimed is:

1. A skin-attachable thin film patch, the thin path comprising:
a base material;
a water-soluble non-woven fabric formed over the base material;
a coating liquid applied to the water-soluble non-woven fabric; and
released paper which protects the coating liquid,
wherein the total thickness of the thin film is 50 μm.

2. The skin-attachable thin film patch of claim 1, wherein the basic weight of the patch is 20 gsm or less, and an average diameter of fiber constituting the non-woven fabric is 5 μm.

3. The skin-attachable thin film patch of claim 2, wherein the basic weight of the patch is in the range of 10 to 20 gsm, and an average diameter of fiber constituting the non-woven fabric is in the range of 1 to 5 μm.

4. The skin-attachable thin film patch of claim 1, wherein the water-soluble non-woven fabric is comprised of fiber including water-soluble synthetic polymer selected from a group consisting of polyvinyl alcohol, polyethylene oxide and polyvinyl pyrrolidone, or modified sugar ingredients selected from a group consisting of pullulan and levan.

5. The skin-attachable thin film patch of claim 1, wherein the water-soluble fiber is formed by means of any one of melt spinning, melt blowing, and electrospinning, into the water-soluble non-woven fabric.

6. The skin-attachable thin film patch of claim 1, wherein the coating liquid comprises 20 to 50% by weight of a moisturizer, 10 to 40% by weight of purified water, 10 to 30% by weight of a thickener, 5 to 10% by weight of oil, and 2% by weight or less of additives.

7. The skin-attachable thin film patch of claim 6, wherein the moisturizer comprises glycerin or diglycerin

8. The skin-attachable thin film patch of claim 6, wherein the thickener is starch or cellulose gum

9. A skin-attachable thin film patch, comprising:
a water-soluble non-woven fabric; and
a coating liquid which has at least one functional substance and is applied to the water-soluble non-woven fabric,
wherein the water-soluble non-woven fabric has an electrically conductive material deposited thereon to have electrical conductivity

10. The skin-attachable thin film patch of claim 9, wherein the water-soluble non-woven fabric is a non-woven fabric produced by means of electrospinning of polyvinyl alcohol.
